(19) United States
(12) Patent Application Publication
(10) Pub. No.: US 2008/0200703 A1
(43) Pub. Date: Aug. 21, 2008

(54) Heterogeneous Ruthenium Catalyst and Method For Hydrogenating a Carboxylic Aromatic Group, in Particular For Producing Core Hydrogenated Bisglycidyl Ether Bisphenols A and F

(75) Inventors: Frederik Van Laar, Dubai (AE); Michael Becker, Offenburg (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/917,635

(22) PCT Filed: Jun. 21, 2006
(86) PCT No.: PCT/EP2006/063380

§ 371 Date: Dec. 14, 2007

(30) Foreign Application Priority Data
Jun. 22, 2005 (DE) 10 2005 029 294.1
Jan. 16, 2006 (DE) 10 2006 002 180.0

(51) Int. Cl. B01J 21/08; C07D 407/12

(52) U.S. Cl. 549/560; 502/261

(57) ABSTRACT

Heterogeneous ruthenium catalyst which comprises amorphous silicon dioxide as support material and can be produced by single or multiple impregnation of the support material with a solution of a ruthenium salt, drying and reduction, wherein the silicon dioxide support material used has a BET surface area (in accordance with DIN 66131) in the range from 250 to 400 m²/g, a pore volume (in accordance with DIN 66134) in the range from 0.7 to 1.1 ml/g and a pore diameter (in accordance with DIN 66134) in the range from 6 to 12 nm, and

process for hydrogenating a carbocyclic aromatic group to form the corresponding carbocyclic aliphatic group, in particular a process for preparing a bisglycidyl ether of the formula I

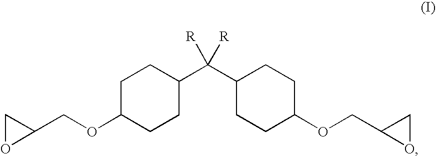

where R is CH₃ or H, by ring hydrogenation of the corresponding aromatic bisglycidyl ether of the formula II

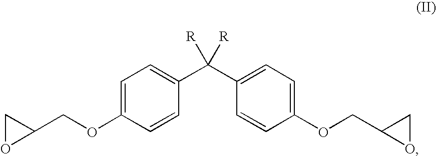

in which the abovementioned heterogeneous ruthenium catalyst is used.

The present invention relates to a heterogeneous ruthenium catalyst which comprises amorphous silicon dioxide as support material and can be produced by single or multiple impregnation of the support material with a solution of a ruthenium salt, drying and reduction, and

a process for catalytically hydrogenating a carbocyclic aromatic group to form the corresponding carbocyclic aliphatic group,
in particular a process for preparing a bisglycidyl ether of the formula I

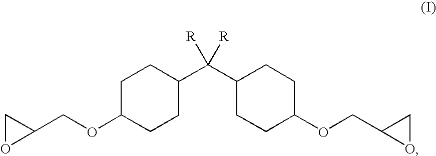

where R is CH₃ or H, by ring hydrogenation of the corresponding aromatic bisglycidyl ether of the formula II

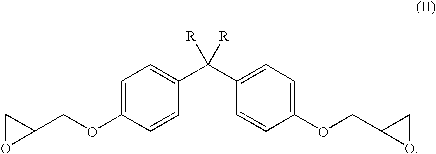

Compound II in which R═H is also referred to as bis[glycidyloxyphenyl]methane (molecular weight: 312 g/mol).
Compound II in which R═CH₃ is also referred to as 2,2-bis[p-glycidyloxyphenyl]propane (molecular weight: 340 g/mol).

The preparation of cycloaliphatic oxirane compounds I which comprise no aromatic groups is of particular interest for the production of light- and weathering-resistant surface coating systems. Such compounds can in principle be prepared by hydrogenation of the corresponding aromatic compounds II. The compounds I are therefore also referred to as “ring-hydrogenated bisglycidyl ethers of bisphenols A and F”.

The compounds II have long been known as constituents of surface coating systems (cf. J. W. Muskopf et al. “Epoxy Resins” in Ullmann's Encyclopedia of Industrial Chemistry, 5th Edition on CD-ROM).

However, the high reactivity of the oxirane groups in the catalytic hydrogenation presents a problem. Under the reaction conditions usually required for the hydrogenation of the aromatic ring, these groups are frequently reduced to alcohols. For this reason, the hydrogenation of the compounds II has to be carried out under very mild conditions. However, this naturally results in a slowing of the desired aromatic hydrogenation.

U.S. Pat. No. 3,336,241 (Shell Oil Comp.) teaches the preparation of cycloaliphatic compounds containing epoxy groups by hydrogenation of corresponding aromatic epoxy compounds using rhodium and ruthenium catalysts. The activity of the catalysts decreases so much after one hydrogenation that the catalyst has to be changed after each hydrogenation in an industrial process. In addition, the selectivity of the catalysts described there leaves something to be desired.

DE-A-36 29 632 and DE-A-39 19 228 (both BASF AG) teach the selective hydrogenation of the aromatic parts of the molecule of bis[glycidyloxyphenyl]methane or of 2,2-bis[p-glycidyloxyphenyl]propane over ruthenium oxide hydrate. This improves the selectivity of the hydrogenation in respect of the aromatic groups to be hydrogenated. However, according to these teachings too, it is advisable to regenerate the catalyst after each hydrogenation, with the separation of the catalyst from the reaction mixture proving to present problems.

EP-A-678 512 (BASF AG) teaches the selective hydrogenation of the aromatic parts of the molecule of aromatic compounds containing oxirane groups over ruthenium catalysts, preferably ruthenium oxide hydrate, in the presence of from 0.2 to 10% by weight of water, based on the reaction mixture. Although the presence of water makes the separation of the catalyst from the reaction mixture easier, it does not alleviate the other disadvantages of these catalysts, e.g. an operating life which is in need of improvement.

EP-A-921 141 and EP-A1-1 270 633 (both Mitsubishi Chem. Corp.) concern the selective hydrogenation of double bonds in particular epoxy compounds in the presence of Rh and/or Ru catalysts having a particular surface area or in the presence of catalysts comprising metals of the platinum group.

JP-A-2002 226380 (Dainippon) discloses the ring hydrogenation of aromatic epoxy compounds in the presence of supported Ru catalysts and a carboxylic ester as solvent.

JP-A2-2001 261666 (Maruzen Petrochem.) relates to a process for the continuous ring hydrogenation of aromatic epoxide compounds in the presence of Ru catalysts which are preferably supported on activated carbon or aluminum oxide.

An article by Y. Hara et al. in Chem. Lett. 2002, pages 1116ff, relates to the “Selective Hydrogenation of Aromatic Compounds Containing Epoxy Group over Rh/Graphite”.

Tetrahedron Lett. 36, 6, pages 885-88, describes the stereoselective ring hydrogenation of substituted aromatics using colloidal Ru.

JP 10-204002 (Dainippon) relates to the use of specific Ru catalysts, in particular Ru catalysts doped with alkali metal, in ring hydrogenation processes.

JP-A-2002 249488 (Mitsubishi) teaches hydrogenation processes in which a supported noble metal catalyst having a chlorine content below 1500 ppm is used.

WO-A1-03/103 830 and WO-A1-04/009 526 (both Oxeno) relate to the hydrogenation of aromatic compounds, in particular the preparation of alicyclic polycarboxylic acids or esters thereof by ring hydrogenation of the corresponding aromatic polycarboxylic acids or esters thereof, and also to catalysts suitable for this purpose.

The processes of the prior art have the disadvantage that the catalysts used have only short operating lives and generally have to be regenerated in a costly fashion after each hydrogenation. The activity of the catalysts also leaves something to be desired, so that only low space-time yields, based on the catalyst used, are obtained under the reaction conditions required for a selective hydrogenation. However, this is not economically justifiable in view of the high cost of ruthenium and thus of the catalyst.

EP-A2-814 098 (BASF AG) relates to, inter alia, processes for the ring hydrogenation of organic compounds in the presence of specific supported Ru catalysts.

WO-A2-02/100 538 (BASF AG) describes a process for preparing particular cycloaliphatic compounds which have side chains containing epoxide groups by heterogeneously catalytic hydrogenation of a corresponding compound which comprises at least one carbocyclic, aromatic group and at least one side chain comprising at least one epoxide group over a ruthenium catalyst.

The ruthenium catalyst is obtainable by

i) treating a support material based on amorphous silicon dioxide one or more times with a halogen-free aqueous solution of a low molecular weight ruthenium compound and subsequently drying the treated support material at a temperature below 200° C.,
ii) reducing the solid obtained in i) by means of hydrogen at a temperature in the range from 100 to 350° C.,
with step ii) being carried out immediately after step i).
WO-A2-02/100538 teaches that the compounds used can “be either monomeric compounds or oligomeric or polymeric compounds” (page 9 above).

The two earlier patent applications PCT/EP/04/014454 and PCT/EP/04/014455, each of Dec. 18, 2004 (both BASF AG), relate to specific Ru catalysts and their use in hydrogenation processes.

It was an object of the present invention to provide an improved selective process for the hydrogenation of aromatic groups to the corresponding “ring-hydrogenated” groups, by means of which high yields and space-time yields [amount of product/(catalyst volume•time)] (kg/(I•h)), [amount of product/(reactor volume•time)] (kg/(I_{reactor}•h)), based on the catalyst used, can be achieved and in which the catalysts used can be used for hydrogenations a number of times without work-up and the catalysts used make stable continuous ring hydrogenation possible. In particular, catalyst operating lives which are higher than those in the process of WO-A2-02/100 538 ought to be achieved.

We have accordingly found a heterogeneous ruthenium catalyst which comprises amorphous silicon dioxide as support material and can be produced by single or multiple impregnation of the support material with a solution of a ruthenium salt, drying and reduction, wherein the silicon dioxide support material used has a BET surface area (in accordance with DIN 66131) in the range from 250 to 400 m²/g, a pore volume (in accordance with DIN 66134) in the range from 0.7 to 1.1 ml/g and a pore diameter (in accordance with DIN 66134) in the range from 6 to 12 nm, and

a process for hydrogenating a carbocyclic aromatic group to form the corresponding carbocyclic aliphatic group, in particular a process for preparing the bisglycidyl ethers of the formula I

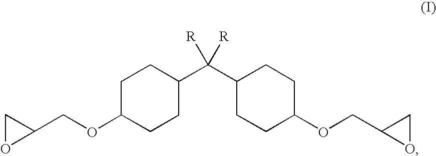

where R is CH₃ or H, by ring hydrogenation of the corresponding aromatic bisglycidyl ether of the formula II

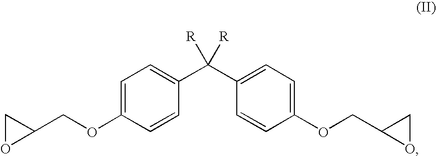

wherein the abovementioned heterogeneous ruthenium catalyst is used.

An important constituent of the catalysts of the invention is the support material based on amorphous silicon dioxide. In this context, the term “amorphous” means that the proportion of crystalline silicon dioxide phases in the support material is less than 10% by weight. However, the support materials used for producing the catalysts can display superstructures formed by a regular arrangement of pores in the support material. (cf., for example, O. W. Flörke, “Silica” in Ullmann's Encyclopedia of Industrial Chemistry 6th Edition on CD-ROM).

Possible support materials are amorphous silicon dioxides comprising at least 90% by weight of silicon dioxide, with the remaining 10% by weight, preferably not more than 5% by weight, of the support material also being able to be another oxidic material, e.g. MgO, CaO, TiO₂, ZrO₂, Fe₂O₃ and/or an alkali metal oxide.

In a preferred embodiment of the invention, the support material is halogen-free, in particular chlorine-free, i.e. the halogen content of the support material is less than 500 ppm by weight, e.g. in the range from 0 to 400 ppm by weight.

Preference is given to support materials which have a specific surface area in the range from 290 to 370 m²/g, preferably from 300 to 360 m²/g, preferably from 310 to 355 m²/g (BET surface area in accordance with DIN 66131).

Suitable amorphous support materials based on silicon dioxide are commercially available:

Particular preference is given to Siliperl AF 125 (Perikat 97) from Engelhard.

Depending on the way in which the process of the invention is carried out, the support material can have various forms. If the process is carried out as a suspension process, the support material will usually be used in the form of finely divided powder for producing the catalysts of the invention. The powder preferably has particle sizes in the range from 1 to 200 μm, in particular from 1 to 100 μm. When the catalyst is used in fixed beds, it is usual to employ shaped bodies made of the support material which are obtainable, for example, by extrusion, ram extrusion or tableting and can have, for example, the shape of spheres, pellets, cylinders, extrudates, rings or hollow cylinders, stars and the like. The dimensions of these shaped bodies are usually in the range from 1 mm to 25 mm. Catalyst extrudates having extrudate diameters of from 1.5 to 5 mm and extrudate lengths of from 2 to 25 mm are frequently used.

The silicon dioxide support material is particularly preferably used in the form of spherical shaped bodies for producing the catalyst.

The spherical shaped bodies preferably have a diameter in the range from 1 to 6 mm, more preferably from 2 to 5.5 mm, in particular from 3 to 5 mm.

The shaped bodies, in particular the spherical shaped bodies, preferably have a (lateral) compressive strength of >60 newton (N), preferably >70 N, more preferably >80 N, more preferably >100 N, e.g: in the range from 90 to 150 N.

To determine the (lateral) compressive strength, the catalyst pellet was, for example, loaded on the cylindrical surface with increasing force between two parallel plates or, for example, the catalyst sphere was loaded with increasing force between two parallel plates until fracture occurred. The force recorded on fracture is the (lateral) compressive strength. The determination was carried out on a test instrument from Zwick, Ulm, having a fixed turntable and a freely movable, vertical punch which pressed the shaped body against the fixed turntable. The freely movable punch was connected to a load cell for recording the force. The instrument was controlled by means of a computer which recorded and evaluated the measured values. 25 shaped bodies which were in good condition (e.g. without cracks and, if appropriate, without broken edges) were taken from a well-mixed catalyst sample, their (lateral) compressive strength was determined and subsequently averaged.

The silicon dioxide support material used for producing the catalyst particularly preferably has a pore volume (DIN 66134) in the range from 0.75 to 1.0 ml/g, particularly preferably from 0.80 to 0.96 ml/g, e.g. from 0.85 to 0.95 ml/g.

Furthermore, the silicon dioxide support material used for producing the catalyst preferably has a pore diameter (in accordance with 66134) in the range from 8 to 10 nm, e.g. in the range from 8.2 to 9.8 nm, in particular in the range from 8.3 to 9.0 nm.

The ruthenium content of the catalysts is preferably in the range from 0.5 to 4% by weight and in particular in the range from 1 to 3% by weight, e.g. from 1.5 to 2.5% by weight, in each case based on the weight of the silicon dioxide support material and calculated as elemental ruthenium (for method of determination, see below).

The catalyst of the invention particularly preferably comprises no Cu, Co, Zn, Rh, Pd, Os, Ir, Hg, Cd, Pb, Bi and/or Pt.

The ruthenium catalysts of the invention are generally produced by firstly treating the selected support material with a solution of a low molecular weight ruthenium compound, hereinafter referred to as (ruthenium) precursor, in such a way that the desired amount of ruthenium is taken up by the support material. Preferred solvents here are glacial acetic acid, water or mixtures thereof. This step will hereinafter also be referred to as impregnation. The support which has been treated in this way is subsequently dried with the abovementioned upper limit to the temperature being adhered to. If appropriate, the solid obtained in this way is then treated again with the aqueous solution of the ruthenium precursor and dried again. This procedure is repeated until the amount of ruthenium compound taken up by the support material corresponds to the desired ruthenium content of the catalyst.

The treatment or impregnation of the support material can be carried out in various ways and depends in a known manner on the shape of the support material. For example, the support material can be sprayed or flushed with the precursor solution or the support material can be suspended in the precursor solution. For example, the support material can be suspended in the aqueous solution of the ruthenium precursor and filtered off from the aqueous supernatant liquid after a particular time. The ruthenium content of the catalyst can then be controlled in a simple fashion via the amount of liquid taken up and the ruthenium concentration of the solution. The impregnation of the support material can, for example, also be carried out by treating the support with a defined amount of the solution of the ruthenium precursor corresponding to the maximum amount of liquid which can be taken up by the support material. For this purpose, the support material can, for example, be sprayed with the required amount of liquid. Suitable apparatuses for this purpose are the apparatuses customarily used for mixing liquids with solids (cf. Vauck/Müller, Grundoperationen chemischer Verfahrenstechnik, 10th edition, Deutscher Verlag für Grundstoffindustrie, 1994, p. 405 ff.), for example tumble dryers, impregnation drums, drum mixers, blade mixers and the like. Monolithic supports are usually flushed with the aqueous solutions of the ruthenium precursor.

The solutions used for impregnation are preferably low in halogen, in particular low in chlorine, i.e. they comprise no halogen or less than 500 ppm by weight, in particular less than 100 ppm by weight, of halogen, e.g. from 0 to <80 ppm by weight of halogen, based on the total weight of the solution. Ruthenium precursors used are therefore RuCl₃ and preferably ruthenium compounds, in particular ruthenium (III) or ruthenium (IV) salts, which comprise no chemically bound halogen and are sufficiently soluble in the solvent. These include, for example, ruthenium(III) nitrosyl nitrate (Ru(NO)(NO₃)₃), ruthenium(III) acetate and also alkali metal ruthenates(IV), e.g. sodium and potassium ruthenate(IV).

A very particularly preferred Ru precursor is Ru(III) acetate. This Ru compound is usually employed as a solution in acetic acid or glacial acetic acid, but it can also be used as a solid. The catalyst of the invention can be produced without using water.

Many ruthenium precursors are commercially available as solutions, but the corresponding solids can also be used. These precursors can be dissolved or diluted using the same component as the solvent supplied, e.g. nitric acid, acetic acid, hydrochloric acid, or preferably using water. Mixtures of water or solvent comprising up to 50% by volume of one or more organic solvents which are miscible with water or solvents, e.g. mixtures with C₁-C₄-alkanols such as methanol, ethanol, n-propanol or isopropanol, can also be used. All mixtures should be chosen so that a single solution or phase is present. The concentration of the ruthenium precursor in the solutions naturally depends on the amount of ruthenium precursor to be applied and on the uptake capacity of the support material for the solution and is generally in the range from 0.1 to 20% by weight.

Drying can be carried out by the customary methods of solids drying with the abovementioned upper limits to the temperature being adhered to. Adherence to the upper limit according to the invention to the drying temperatures is important for the quality, i.e. the activity, of the catalyst. Exceeding the abovementioned drying temperatures leads to a significant loss in activity. Calcination of the support at higher temperatures, e.g. above 300° C. or even 400° C., as is proposed in the prior art, is not only superfluous but also has an adverse effect on the activity of the catalyst. To achieve satisfactory drying rates, drying is preferably carried out at elevated temperature, preferably at ≦180° C., particularly preferably at ≦1 60° C., and at at least 40° C., in particular at least 70° C., especially at least 100° C., very particularly preferably at least 140° C.

Drying of the solid impregnated with the ruthenium precursor is usually carried out under atmospheric pressure, although a reduced pressure can also be employed to promote drying. A gas stream, e.g. air or nitrogen, will frequently be passed over or through the material to be dried in order to promote drying.

The drying time naturally depends on the desired degree of drying and on the drying temperature and is generally in the range from 1 hour to 30 hours, preferably in the range from 2 to 10 hours.

Drying of the treated support material is preferably carried out to the point where the content of water or of volatile solvent constituents prior to the subsequent reduction is less than 5% by weight, in particular not more than 2% by weight, based on the total weight of the solid. The proportions by weight indicated correspond to the weight loss of the solid determined at a temperature of 160° C., a pressure of 1 bar and a time of 10 minutes. In this way, the activity of the catalysts used according to the invention can be increased further.

Drying is preferably carried out with the solid which has been treated with the precursor solution being kept in motion, for example by drying the solid in a rotary tube oven or a rotary sphere oven. In this way, the activity of the catalysts of the invention can be increased further.

The conversion of the solid obtained after drying into its catalytically active form is achieved by reducing the solid in a manner known per se at the temperatures indicated above.

For this purpose, the support material is brought into contact with hydrogen or a mixture of hydrogen and an inert gas at the temperatures indicated above. The absolute hydrogen pressure is of minor importance for the result of the reduction and will generally be in the range from 0.2 bar to 1.5 bar. The hydrogenation of the catalyst material is frequently carried out at a hydrogen pressure of one atmosphere in a stream of hydrogen. The reduction is preferably carried out with the solid being kept in motion, for example by reducing the solid in a rotary tube oven or a rotary sphere oven. In this way, the activity of the catalysts of the invention can be increased further.

The reduction can also be carried out by means of organic reducing agents such as hydrazine, formaldehyde, formates or acetates.

After the reduction, the catalyst can be passivated in a known manner, e.g. by briefly treating the catalyst with an oxygen-comprising gas, e.g. air, but preferably with an inert gas mixture comprising from 1 to 10% by volume of oxygen, to improve the handleability. CO₂ or CO₂/O₂ mixtures can also be employed here.

The active catalyst can also be stored under an inert organic solvent, e.g. ethylene glycol.

As a result of the way in which the catalysts of the invention are produced, the ruthenium is present as metallic ruthenium in these catalysts. Furthermore, electron-microscopic studies (SEM or TEM) have shown that a surface-impregnated catalyst is present: the ruthenium concentration within a catalyst particle decreases from the outside toward the interior, with a ruthenium layer being present at the surface of the particle. In preferred cases, crystalline ruthenium can be detected in the outer shell by means of SAD (selected area diffraction) and XRD (X-ray diffraction).

In the catalyst shell, the Ru is, in particular, present in aggregated-agglomerated form; in the catalyst core, the ruthenium concentration is at its lowest (the size of the ruthenium particles in the core is, for example, in the range 1-2 nm).

In a particularly preferred variant, the ruthenium in the shell and in the core is present in finely dispersed form.

The average dispersity of the ruthenium in the catalyst is preferably in the range from 30 to 60%, in particular in the range from 40 to 50% (in each case measured by means of CO sorption in accordance with DIN 66136-3, cf. below).

In addition, as a result of the use of halogen-free, in particular chlorine-free, ruthenium precursors and solvents in the production of the catalysts of the invention, their halide content, in particular chloride content, is below 0.05% by weight (from 0 to <500 ppm by weight, e.g. in the range 0-400 ppm by weight), based on the total weight of the catalyst.

The chloride content is, for example, determined by the ion-chromatographic method described below.

In this document, all ppm figures are by weight (ppm by weight) unless indicated otherwise.

The support material preferably comprises not more than 1% by weight and in particular not more than 0.5% by weight and in particular <500 ppm by weight of aluminum oxide, calculated as Al₂O₃.

Since the condensation of the silica can also be influenced by aluminum and iron, the total concentration of Al(III) and Fe(II and/or III) is preferably less than 300 ppm, particularly preferably less than 200 ppm, and is, for example, in the range from 0 to 180 ppm.

The alkali metal oxide content generally results from the production of the support material and can be up to 2% by weight. It is frequently less than 1% by weight. Supports which are free of alkali metal oxide (from 0 to <0.1% by weight) are also suitable. The proportion of MgO, CaO, TiO₂ or ZrO₂ can amount to up to 10% by weight of the support material and is preferably not more than 5% by weight. However, support materials which comprise no detectable amounts of these metal oxides (from 0 to <0.1% by weight) are also suitable.

Since Al(III) and Fe(II and/or III) incorporated in silica can produce acid centers, it is preferred that charge-compensating cations, preferably alkaline earth metal cations (M²⁺, M=Be, Mg, Ca, Sr, Ba), are present in the support. This means that the weight ratio of M(II) to (Al(III)+Fe(II and/or III)) is greater than 0.5, preferably >1, particularly preferably greater than 3.

The Roman numbers in brackets after the element symbol indicate the oxidation state of the element.

The Ru catalyst of the invention after reduction particularly preferably also has the following features:

N₂ sorption:
BET (DIN 66131): in the range from 250 to 400 m²/g, particularly preferably from 290 to 380 m²/g, very particularly preferably from 310 to 375 m²/g, more particularly preferably from 320 to 370 m²/g, in particular from 340 to 360 m²/g, e.g. from 344 to 357 m²/g,
pore volume (DIN 66134): in the range from 0.75 to 0.90 ml/g, in particular from 0.80 to 0.89 ml/g, e.g. from 0.81 to 0.88 ml/g or from 0.85 to 0.87 ml/g,
pore diameter (4V/A) (DIN 66134): from 7.5 to 10 nm, in particular from 7.8 to 9.5 nm, e.g. from 8.0 to 9.0 nm, from 8.1 to 8.7 nm or from 8.2 to 8.5 nm.

Hg porosimetry (DIN 66133):

pore volume: in the range from 0.70 to 0.91 ml/g, in particular from 0.75 to 0.90 ml/g, e.g. from 0.76 to 0.89 ml/g, from 0.80 to 0.88 ml/g or from 0.82 to 0.87 ml/g.
pore diameter (4V/A): from 8 to 11 nm, in particular from 9 to 10.5 nm, e.g. from 9.3 to 10.0 nm.

The carbocyclic aromatic group in the organic compound to be hydrogenated is in particular a benzene ring, which may bear substituents.

Examples of compounds comprising a benzene ring which are able to be hydrogenated by the process of the invention to form the corresponding compound comprising a saturated carbocyclic 6-membered ring are listed in the following table:

| Starting material | Product |
|---|---|
| Benzene | Cyclohexane |
| Toluene | Methylcyclohexane |
| Ethylbenzene | Ethylcyclohexane |
| Xylene (o-, m- or p-) or isomer mixture | Dimethylcyclohexane |
| Phenol | Cyclohexanol |
| Alkyl-substituted phenols, e.g. C₁₋₁₀- | Alkyl-substituted |
| alkylphenol such as 4-tert-butylphenol, | cyclohexanols, e.g. C₁₋₁₀- |
| 4-nonylphenol | alkylcyclohexanol |
| Bis(p-hydroxyphenyl)methane | Bis(4-hydroxycyclo- |
| | hexyl)methane |
| Bis(p-hydroxyphenyl)dimethylmethane | Bis(4-hydroxycyclo- |
| | hexyl)dimethylmethane |
| Aniline | Cyclohexylamine |
| C₁₋₁₀-Alkyl-substituted aniline | C₁₋₁₀-Alkyl-substituted |
| | cyclohexylamine |
| N,N-di-C₁₋₁₀-Alkylaniline | N,N-di-C₁₋₁₀-Alkyl- |
| | cyclohexylamine |
| Diaminobenzene | Diaminocyclohexane |
| Bis(p-aminophenyl)methane | Bis(4-aminocyclo- |
| | hexyl)methane |

As starting compounds for the hydrogenation process of the invention, mention may also be made by way of example of the following substance classes and materials:

reaction products of bisphenol A or bisphenol F or comparable alkylene- or cycloalkylene-bridged bisphenol compounds with epichlorohydrin. Bisphenol A or bisphenol F or comparable compounds can be reacted with epichlorohydrin and bases in a known manner (e.g. Ullmann's Encyclopedia of Industrial Chemistry, 5th Edition, VCH (1987), Vol. A9, p. 547) to give glycidyl ethers of the general formula IIa,

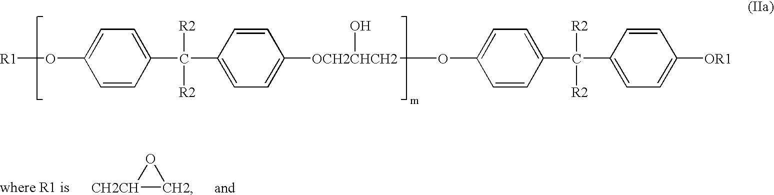

R² is hydrogen or a C₁-C₄-alkyl group, e.g. methyl, or two radicals R² bound to one carbon atom form a C₃-C₅-alkylene group, and m is from zero to 40.

Phenol and cresol epoxy novolaks IIb

Novolaks of the general formula IIb can be obtained by acid-catalyzed reaction of phenol and cresol and conversion of the reaction products into the corresponding glycidyl ethers (cf., for example, bis[4-(2,3-epoxypropoxy)phenyl]methane):

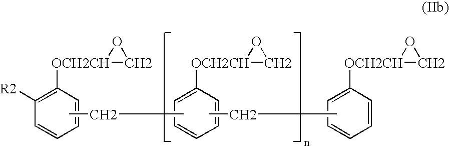

where R² is hydrogen or a methyl group and n is from 0 to 40 (cf. J. W. Muskopf et al. “Epoxy Resins 2.2.2” in Ullmann's Encyclopedia of Industrial Chemistry, 5th Edition on CD-ROM).

Glycidyl ethers of reaction products of phenol and an aldehyde:

Acid-catalyzed reaction of phenol and aldehydes and subsequent reaction with epichlorohydrin makes it possible to obtain glycidyl ethers, e.g. 1,1,2,2-tetrakis[4-(2,3-epoxypropoxy)phenyl]ethane can be obtained from phenol and glyoxal (cf. J. W. Muskopf et al. “Epoxy Resins 2.2.3” in Ullmann's Encyclopedia of Industrial Chemistry, 5th Edition on CD-ROM).

Glycidyl ethers of phenol-hydrocarbon novolaks, e.g. 2,5-bis[(glycidyloxy)phenyl]octahydro-4,7-methano-5H-indene and its oligomers.

Aromatic glycidyl amines:

Examples which may be mentioned are the triglycidyl compound of p-aminophenol, 1-(glycidyloxy)-4-[N,N-bis(glycidyl)amino]benzene, and the tetraglycidyl compound of methylenediamine, bis{4-[N,N-bis(2,3-epoxypropyl)amino]phenyl}methane.

Further specific examples are: tris[4-(glycidyloxy)phenyl]methane isomers and glycidyl esters of aromatic monocarboxylic, dicarboxylic and tricarboxylic acids, e.g. diglycidyl phthalates and isophthalates.

In a particular embodiment of the process of the invention, aromatic bisglycidyl ethers of the formula II

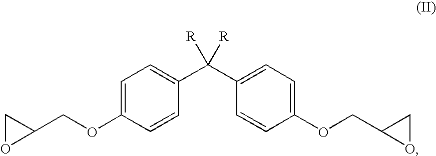

where R is CH₃ or H, are ring hydrogenated.

Preferred aromatic bisglycidyl ethers of the formula II have a content of chloride and/or organically bound chlorine of ≦1000 ppm by weight, preferably in the range from 0 to <1000 ppm, e.g. from 100 to <950 ppm by weight.

The content of chloride and/or organically bound chlorine is, for example, determined ion-chromatographically or coulometrically using the methods described below.

According to a particular embodiment of this process variant according to the invention, it has been recognized that it is, surprisingly, also advantageous for the aromatic bisglycidyl ether of the formula II which is used to have a content of corresponding oligomeric bisglycidyl ethers of less than 10% by weight, in particular less than 5% by weight, particularly preferably less than 1.5% by weight, very particularly preferably less than 0.5% by weight, e.g. in the range from 0 to <0.4% by weight.

According to this particular embodiment of this process variant according to the invention, it has been found that the oligomer content of the feed has a critical influence on the operating life of the catalyst, i.e. the conversion remains at a high level for longer. When a bisglycidyl ether II which has, for example, been distilled and is therefore low in oligomers is used, a slowed catalyst deactivation compared to a corresponding commercial standard product (e.g.: ARALDIT GY 240 BD from Vantico) is observed.

The oligomer content of the aromatic bisglycidyl ethers of the formula II which are used is preferably determined by GPC measurement (gel permeation chromatography) or by determination of the evaporation residue.

The evaporation residue is determined by heating the aromatic bisglycidyl ether for 2 hours at 200° C. and for a further 2 hours at 300° C., in each case at 3 mbar.

For the further respective conditions for determining the oligomer content, see below.

The respective oligomeric bisglycidyl ethers generally have a molecular weight determined by GPC in the range from 380 to 1500 g/mol and possess, for example, the following structures (cf., for example, Journal of Chromatography 238 (1982), pages 385-398, page 387):

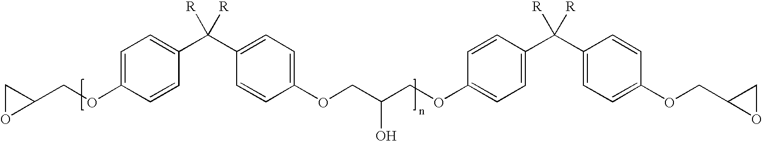

R═CH₃ or H. n=1, 2, 3 or 4.

The respective oligomeric bisglycidyl ethers have a molecular weight in the range from 568 to 1338 g/mol, in particular from 568 to 812 g/mol, when R═H, and have a molecular weight in the range from 624 to 1478 g/mol, in particular from 624 to 908 g/mol, when R═CH₃.

The removal of the oligomers is carried out, for example, by means of chromatography or, on a relatively large scale, preferably by distillation, e.g. in a batch distillation on the laboratory scale or in a thin film evaporator, preferably in a short path distillation, on an industrial scale, in each case under reduced pressure.

In a batch distillation for the removal of oligomers at, for example, a pressure of 2 mbar, the bath temperature is about 260° C. and the temperature at which the distillate goes over at the top is about 229° C.
The removal of oligomers can likewise be carried out under milder conditions, for example under reduced pressures in the range from 1 to 10⁻³ mbar. At a working pressure of 0.1 mbar, the boiling point of the oligomer-comprising starting material is reduced by 20-30° C. depending on the starting material, and the thermal stress on the product is thus also reduced. To minimize the thermal stress, the distillation is preferably carried out continuously in a thin film evaporator or particularly preferably in a short path evaporator.

In the process of the invention, the hydrogenation of the starting materials, e.g. the compounds II, preferably occurs in the liquid phase. The hydrogenation can be carried out in the absence of solvents or in an organic solvent. Owing to the sometimes high viscosity of the compounds II, they are preferably used as a solution or mixture in an organic solvent.

Possible organic solvents are basically those which are able to dissolve the starting material, e.g. the compound II, virtually completely or are completely miscible with this and are inert under the hydrogenation conditions, i.e. are not hydrogenated.

Examples of suitable solvents are cyclic and acyclic ethers, e.g. tetrahydrofuran, dioxane, methyl tert-butyl ether, dimethoxyethane, dimethoxypropane, dimethyl diethylene glycol, aliphatic alcohols such as methanol, ethanol, n-propanol or isopropanol, n-, 2-, iso- or tert-butanol, carboxylic esters such as methyl acetate, ethyl acetate, propyl acetate or butyl acetate, and also aliphatic ether alcohols such as methoxypropanol.

The concentration of starting material, e.g. of compound II, in the liquid phase to be hydrogenated can in principle be chosen freely and is frequently in the range from 20 to 95% by weight, based on the total weight of the solution/mixture. In the case of starting materials which are sufficiently fluid under the reaction conditions, the hydrogenation can also be carried out in the absence of a solvent.

Apart from carrying out the reaction (hydrogenation) under water-free conditions, it has been found to be useful in a number of cases to carry out the reaction (hydrogenation) in the presence of water. The proportion of water can be, based on the mixture to be hydrogenated, up to 10% by weight, e.g. from 0.1 to 10% by weight, preferably from 0.2 to 7% by weight and in particular from 0.5 to 5% by weight.

The actual hydrogenation is usually carried out by a method analogous to the known hydrogenation processes as are described in the prior art mentioned at the outset. For this purpose, the starting material, e.g. the compound II, preferably as a liquid phase, is brought into contact with the catalyst in the presence of hydrogen. The catalyst can either be suspended in the liquid phase (suspension process) or the liquid phase is passed over a moving bed of catalyst (moving-bed process) or a fixed bed of catalyst (fixed-bed process). The hydrogenation can be carried out either continuously or batchwise. The process of the invention is preferably carried out as a fixed-bed process in trickle-bed reactors. The hydrogen can be passed over the catalyst either in cocurrent with or in countercurrent to the solution of the starting material to be hydrogenated.

Suitable apparatuses for carrying out a hydrogenation in the suspension mode and also for hydrogenation over a moving bed of catalyst or a fixed bed of catalyst are known from the prior art, e.g. from Ullmanns Enzyklopädie der Technischen Chemie, 4th edition, Volume 13, p. 135 ff. and also from P. N. Rylander, “Hydrogenation and Dehydrogenation” in Ullmann's Encyclopedia of Industrial Chemistry, 5th ed. on CD-ROM.

The hydrogenation of the invention can be carried out either at a hydrogen pressure of one atmosphere or at a superatmospheric pressure of hydrogen, e.g. an absolute hydrogen pressure of at least 1.1 bar, preferably at least 10 bar. In general, the absolute hydrogen pressure will not exceed 325 bar and preferably 300 bar. The absolute hydrogen pressure is particularly preferably in the range from 20 to 300 bar, e.g. in the range from 50 to 280 bar.

The reaction temperatures in the process of the invention are generally at least 30° C. and will frequently not exceed a value of 200° C. In particular, the hydrogenation process is carried out at temperatures in the range from 40 to 150° C., e.g. from 40 to 100° C., and particularly preferably in the range from 45 to 80° C.

Possible reaction gases are hydrogen and also hydrogen-comprising gases which comprise no catalyst poisons such as carbon monoxide or sulfur-comprising gases, e.g. mixtures of hydrogen with inert gases such as nitrogen or offgases from a reformer, which usually further comprise volatile hydrocarbons. Preference is given to using pure hydrogen (purity ≧99.9% by volume, preferably ≧99.95% by volume, in particular ≧99.99% by volume).

Owing to the high catalyst activity, comparatively small amounts of catalyst, based on the starting material used, are required. Thus, less than 5 mol %, e.g. from 0.2 mol % to 2 mol %, of ruthenium will generally be used per 1 mol of starting material in a suspension process carried out batchwise. When the hydrogenation is carried out continuously, the starting material to be hydrogenated will usually be passed over the catalyst in an amount of from 0.05 to 3 kg/(l(catalyst).h), in particular from 0.15 to 2 kg/(l(catalyst).h).

Of course, when the activity of the catalysts used in this process drops, they can be regenerated by the customary methods known to those skilled in the art for noble metal catalysts such as ruthenium catalysts. Mention may here be made of, for example, treatment of the catalyst with oxygen as described in BE 882 279, treatment with dilute, halogen-free mineral acids as described in U.S. Pat. No. 4,072,628, or treatment with hydrogen peroxide, e.g. in the form of aqueous solutions having a concentration of from 0.1 to 35% by weight, or treatment with other oxidizing substances, preferably in the form of halogen-free solutions. The catalyst is usually rinsed with a solvent, e.g. water, after the reactivation and before renewed use.

In the hydrogenation process of the invention, the aromatic rings of the bisglycidyl ether of the formula II

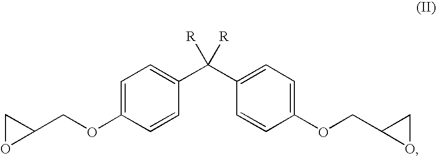

where R is CH₃ or H, are preferably hydrogenated completely, with the degree of hydrogenation being >98%, very particularly preferably >98.5%, e.g. >99.0%, in particular >99.5%, e.g. in the range from >99.8 to 100%.

The degree of hydrogenation (Q) is defined by

Q (%)=([number of cycloaliphatic C6 rings in the product]/[number of aromatic C6 rings in the starting material])•100

The ratio, e.g. molar ratio, of the cycloaliphatic C6 rings to aromatic C6 rings is preferably determined by means of ¹H-NMR spectroscopy (integration of the aromatic and corresponding cycloaliphatic ¹H signals).

Bisglycidyl ethers of the formula I

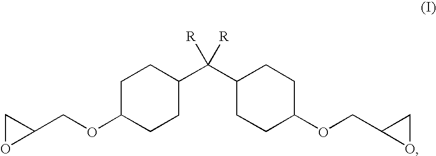

where R is CH₃ or H, can advantageously be prepared by the hydrogenation process of the invention.

The bisglycidyl ethers of the formula I preferably have a content of corresponding oligomeric ring-hydrogenated bisglycidyl ethers of the formula

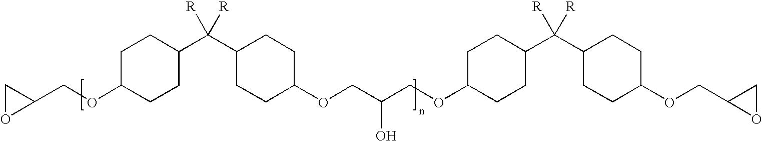

(where R is CH₃ or H) having n=1, 2, 3 or 4, of less than 10% by weight, preferably less than 5% by weight, in particular less than 1.5% by weight, very particularly preferably less than 0.5% by weight, e.g. in the range from 0 to <0.4% by weight.

The content of oligomeric ring-hydrogenated bisglycidyl ethers is preferably determined by heating the aromatic bisglycidyl ether for 2 hours at 200° C. and for a further 2 hours at 300° C., in each case at 3 mbar, or by GPC measurement (gel permeation chromatography).

For the further respective conditions for determining the oligomer content, see below.

The bisglycidyl ethers of the formula I preferably have a total chlorine content determined in accordance with DIN 51408-2 of ≦1000 ppm by weight, in particular in the range from 0 to <1000 ppm by weight, e.g. in the range from 100 to <950 ppm by weight.

The bisglycidyl ethers of the formula I preferably have a ruthenium content determined by mass spectrometry with inductively coupled plasma (ICP-MS) of less than 0.3 ppm by weight, in particular less than 0.2 ppm by weight, very particularly preferably less than 0.15 ppm by weight, e.g. in the range from 0 to 0.1 ppm by weight.

The bisglycidyl ethers of the formula I preferably have a platinum-cobalt color number (APHA color number) determined in accordance with DIN EN ISO 6271-2 of less than 30, in particular less than 25, e.g. in the range from 1 to 24.

The bisglycidyl ethers of the formula I preferably have an epoxy equivalent weight determined in accordance with the standard ASTM-D-1652-88 in the range from 170 to 240 g/equivalent, in particular in the range from 175 to 230 g/equivalent, very particularly preferably in the range from 180 to 225 g/equivalent.

The bisglycidyl ethers of the formula I preferably have a content of hydrolyzable chlorine determined in accordance with DIN 53188 of less than 500 ppm by weight, in particular less than 400 ppm by weight, very particularly preferably less than 350 ppm by weight, e.g. in the range from 0 to 300 ppm by weight.

The bisglycidyl ethers of the formula I preferably have a kinematic viscosity determined in accordance with DIN 51562 Part 1 of less than 900 mm²/s, in particular less than 850 mm²/s, e.g. in the range from 400 to 800 mm²/s, in each case at 25° C.

The bisglycidyl ethers of the formula I preferably have a cis/cis:cis/trans:trans/trans isomer ratio in the range 44-63%:34-53%:3-22%.

The cis/cis:cis/trans:trans/trans isomer ratio is particularly preferably in the range 46-60%:36-50%:4-18%.
The cis/cis:cis/trans:trans/trans isomer ratio is very particularly preferably in the range 48-57%:38-47%:5-14%.
In particular, the cis/cis:cis/trans:trans/trans isomer ratio is in the range 51-56%:39-44%:5-10%.

The bisglycidyl ethers of the formula I are particularly preferably obtained by complete hydrogenation of the aromatic rings of a bisglycidyl ether of the formula II

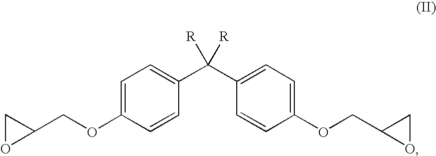

where R is CH3 or H, with the degree of hydrogenation being >98%, very particularly preferably >98.5%, e.g. >99.0%, in particular >99.5%, e.g. in the range from >99.8 to 100%.

### EXAMPLES

Production of a Catalyst According to the Invention

100 g of Siliperl AF 125 (3-5 mm spheres, Engelhard, Lot 2960211: (lateral) compressive strength: 76 N (for measurement method, see above), BET: 353 m²/g (in accordance with DIN 66131), pore volume: 0.95 ml/g and mean pore diameter: 8.6 nm (both in accordance with DIN 66134)) having a water uptake of 9.7 ml/10 g of support were placed in a vessel/dish. 51.83 g of Ru acetate solution (from Umicore, w(Ru)=4.34%, batch no. 0255) were made up to 95 ml with deionized water. This stock solution was distributed over the support and dried overnight at 120° C. (drying oven, in air). The dried product was reduced under hydrogen at 300° C. for 2 hours (25° C.-300° C. in 90 min., using 60 l/h of N₂ then 50 l/h of H₂-10 l/h of N₂). The product was subsequently cooled under nitrogen and passivated by means of diluted air (e.g. using 3 l/h of air-50 l/h of N₂) at room temperature (RT) (T<30° C.). The finished catalyst comprised 2.0% by weight of Ru.

The support can be impregnated by known methods; drying can be carried out with the support either moving or stationary: preference is given to gentle motion taking place or the support being kept in motion at the beginning and dried in a static fashion at the end, so that the ruthenium layer is not abraded off. The reduction can be carried out with the support either moving or stationary. Passivation can be carried out by the method known to those skilled in the art.

Ruthenium content: 2.0% by weight (other catalysts produced by a method based on the above method comprised from 1.6 to 2.5% by weight of Ru)
Method description: from 0.03 to 0.05 gram of the sample is mixed with 5 g of sodium peroxide in an Alsint crucible and slowly heated on a hotplate. The substance/flux mixture is then firstly melted over an open flame and subsequently heated over a blowtorch flame until it is red hot. The fusion is finished as soon as a clear melt has been obtained.
The cooled melt cake is dissolved in 80 ml of water, the solution is heated to boiling (decomposition of H₂O₂) and subsequently, after cooling, admixed with 50 ml of hydrochloric acid.
The solution is then made up to a volume of 250 ml with water.
Measurement: the measurement of this sample solution is carried out by ICP-MS for the isotope Ru 99.

Ru dispersity: 45% (by CO sorption, assumed stoichiometry factor: 1; sample preparation: reduction of the sample by means of hydrogen at 200° C. for 30 minutes and subsequently flushed with helium at 200° C. for 30 minutes—measurement of the metal surface area using pulses of the gas to be adsorbed in an inert gas stream (CO) to saturation chemisorption at 35° C. Saturation is achieved when no more CO is adsorbed, i.e. the areas of 3-4 successive peaks (detector signal) are constant and similar to the peak of a nonadsorbed pulse. Pulse volume is determined to a precision of 1%; pressure and temperature of the gas have to be checked). (Method: DIN 66136-3).

### Hydrogenation Example 1

A heatable double-walled stainless steel reaction tube (length: 0.8 m; diameter: 12 mm) which was charged with 75 ml of the abovementioned catalyst (31 g, 2.0% by weight of Ru on Siliperl AF 125 3-5 mm) and was equipped with a feed pump for introduction of the starting material solution, a separator for separating gas and liquid phases with level regulator, offgas regulator and sampling facility served as reactor. The plant was operated in the upflow mode (i.e. with the flow direction from the bottom upward) without circulation of liquid. The temperature at the beginning (inlet) and at the end (outlet) of the catalyst bed was measured by means of a thermocouple (cf. table below).

In the hydrogenation, a 40% strength by weight solution of distilled low-oligomer bisphenol A bisglycidyl ether (2,2-di[p-glycidoxyphenyl]propane, Epilox A 17-01 from Leuna-Harze, batches 16/03 and 06/04: EEW =172 g/eq.) in stabilizer-free THF which comprised 4.5% by weight of water was used. The hydrogenation was carried out a space velocity of the catalyst of 0.15 kg_{starting material}/L_{catalyst}•h, a temperature of about 44-50° C. (cf. table below), a hydrogen pressure of 250 bar and a hydrogen feed rate of 15 standard l/h (standard l=standard liters=volume at STP). The reactor was operated in the upflow mode.

The conversions, selectivities and ruthenium concentrations in the output from the reactor (after removal of the solvent at 110° C. under a reduced pressure of 10 mbar on a rotary evaporator) which were achieved can be seen in the following table. The figure given for the feed rate is based on the 40% strength solution of the low-oligomer bisphenol A bisglycidyl ether.

| Time of | | | Feed | Conversion | Epoxy equivalent | | Ruthenium |
|---|---|---|---|---|---|---|---|
| operation | Temperature | | rate | (1H-NMR) | weight (EEW) | Selectivity | content |
| [h] | Inlet | Outlet | g/h | % | g/eq. | % | [ppm] |
| 0 | 47 | 44 | 27.4 | | | | |
| 47 | 47 | 44 | 27.4 | 98.1 | 210 | 84.7 | |
| 71 | 47 | 45 | 27.4 | 98.1 | 205 | 86.8 | |
| 143 | 47 | 45 | 27.4 | 95.3 | 200 | 88.9 | |
| 167 | 47 | 45 | 27.4 | 94.3 | 198 | 89.7 | 0.1 |
| 191 | 47 | 45 | 27.4 | 94.1 | 199 | 89.3 | |
| 215 | 47 | 45 | 27.4 | 94.1 | 193 | 92.0 | |
| 239 | 47 | 45 | 27.4 | | | | |
| 311 | 47 | 45 | 27.4 | 90.6 | 196 | 90.5 | 0.1 |
| 335 | 47 | 45 | 27.4 | | | | |
| 359 | 47 | 45 | 27.4 | 91.2 | 195 | 91.0 | |
| 383 | 47 | 45 | 27.4 | | | | |
| 407 | 47 | 45 | 27.4 | 89.9 | 196 | 90.5 | |
| 479 | 47 | 45 | 27.4 | 88.5 | 190 | 93.3 | |
| 551 | 47 | 45 | 27.4 | 88.5 | 194 | 91.4 | |
| 647 | 47 | 45 | 27.4 | 86.6 | 186 | 95.3 | |
| 695 | 47 | 45 | 27.4 | 86.6 | 196 | 90.4 | |
| 784 | 47 | 45 | 27.4 | 82.7 | 191 | 92.7 | |
| 832 | 47 | 45 | 27.4 | 84.6 | 189 | 93.7 | |
| 856 | 50 | 48 | 27.4 | | | | |
| 880 | 50 | 48 | 27.4 | | | | |
| 952 | 50 | 48 | 27.4 | 88.1 | 191 | 92.8 | |
| 1000 | 50 | 48 | 27.4 | 88.1 | | | |
| 1007 | 50 | 48 | 27.4 | | | | |
| 1031 | 50 | 48 | 27.4 | 87.8 | 192 | 92.3 | 0.1 |

The catalyst displayed a constant activity and selectivity over the entire period of the experiment.

### Hydrogenation Example 2

In the experimental setup described in hydrogenation example 1, the partially reacted reaction product mixture (reaction product mixture was partly collected) from hydrogenation example 1 was subjected to an after-hydrogenation over the same catalyst to achieve the desired degree of conversion. The residual aromatics content of the partially hydrogenated product fed in was 10.1% according to H-NMR, corresponding to a conversion of 89.9%. The epoxy equivalent weight was 195 g/equivalent. The hydrogenation was carried out at a temperature of about 44-50° C. (cf. table below), a hydrogen pressure of 250 bar and a hydrogen feed rate of 15 standard l/h (standard l=standard liters=volume at STP). The reactor was operated in the upflow mode.

The conversions, selectivities and ruthenium concentrations in the output from the reactor (after removal of the solvent at 110° C. under a reduced pressure of 10 mbar on a rotary evaporator) which were achieved can be seen in the following table.

The conversion was determined by means of ¹H-NMR (decrease in the signals of the aromatic protons vs. increase in the signals of the aliphatic protons). The conversion reported in the examples is based on the hydrogenation of the aromatic groups.

| Time of | | | Feed | Conversion | Epoxy equivalent | | Ruthenium |
|---|---|---|---|---|---|---|---|
| operation | Temperature | | rate | (1H-NMR) | weight (EEW) | Selectivity | content |
| [h] | Inlet | Outlet | g/h | % | g/eq. | % | [ppm] |
| Starting material (collected output | | | | 89.9 | 195 | 91.0 | |
| from hydrogenation example 1): | | | | | | | |
| 1102 | 49 | 47 | 27.4 | 100.0 | 208 | 85.6 | |
| 1126 | 49 | 47 | 27.4 | | | | |
| 1150 | 49 | 47 | 37.5 | | | | |
| 1174 | 49 | 47 | 37.5 | 98.6 | 203 | 87.6 | <0.2 |
| 1200 | 49 | 47 | 37.5 | | | | |
| 1271 | 51 | 49 | 37.5 | 99.1 | 206 | 86.4 | |
| 1296 | 51 | 49 | 37.5 | | | | |
| 1318 | 51 | 49 | 37.5 | | | | |
| 1343 | 51 | 49 | 37.5 | 98.5 | 204 | 87.2 | |
| 1368 | 51 | 49 | 37.5 | | | | |
| 1439 | 53 | 51 | 37.5 | 99.0 | 205 | 86.8 | |
| 1463 | 53 | 51 | 37.5 | | | | |
| 1487 | 53 | 51 | 37.5 | | | | |
| 1511 | 53 | 51 | 37.5 | 98.7 | 205 | 86.8 | <0.2 |
| 1535 | 53 | 51 | 37.5 | | | | |
| 1607 | 55 | 53 | 37.5 | 99.0 | 206 | 86.4 | <0.2 |
| 1655 | 55 | 53 | 27.4 | | | | |
| 1679 | 55 | 53 | 27.4 | 98.8 | 203 | 87.6 | |

The reaction product mixture generated in hydrogenation example 2 was partly collected, combined and analyzed:
conversion=98.6% (H-NMR), epoxy equivalent weight=204 g/equivalent, selectivity=87%

The previously combined reaction product mixtures from hydrogenation example 2 were freed of the solvent mixture in a thin film evaporator having a glass double wall (area=0.1 m², circumference=0.25 m) under reduced pressure (800 mbar), a temperature of 140° C. (oil temperature in the double wall) and a feed rate of 2500 g/h of the solution. The temperature at which the distillate went over was 85° C. The distillate was condensed in a glass condenser operated using a cooling medium at 15° C. The feed was metered in by means of a metering pump and was regulated by means of a balance. A total of 13.32 kg of reaction product mixture from hydrogenation example 2 were freed of solvent. The output obtained at the bottom was fed by means of a metering pump through a second thin film evaporator having a glass double wall (area=0.046 m², circumference=0.11 m) under reduced pressure (5-10 mbar), a temperature of 140° C. (oil temperature in double wall) to remove residual amounts of solvent and by-products of the hydrogenation, e.g. epoxypropanol, 1,2- and 1,3-propanediol, isopropylcyclohexane.

This gave 5.30 kg of a hydrogenated bisphenol A bisglycidyl ether which had the following properties:

| Residual aromatics content (¹H-NMR): | 98.6% | |
|---|---|---|
| Epoxy equivalent weight (determination | 204 | g/equivalent |
| based on ASTM D1652-88): | | |
| Selectivity: | 87% | |
| Platinum-cobalt color number (determina- | 5 | |
| tion based on DIN EN ISO 6271-2) | | |
| Kinematic viscosity at 25° C. (determina- | 595 | mm²*s⁻¹ |
| tion in accordance with DIN 51562 Part 1): | | |
| Density at 25° C. (determination in | 1.05 | g/ml |
| accordance with DIN 53217 Part 5): | | |
| Ruthenium content (determination by ICP- | 0.1 | ppm (ICP-MS) |
| MS, see below): | | |
| Content of volatile compounds (based on | <2.5% | by weight |
| DIN 16945 4.8) | | |
| Total chlorine content (determination in | <1000 | mg/kg |
| accordance with DIN 51408-2) | | |

### Hydrogenation Example 3

A heatable double-walled stainless steel reaction tube (length: 1.4 m; diameter: 12 mm) which was charged with 90 ml of the abovementioned catalyst (31 g, 2.0% by weight of Ru on Siliperl 3-5 mm) and was equipped with a feed pump for introduction of the starting material solution, a separator for separating gas and liquid phases with level regulator, offgas regulator, liquid recirculation (circuit) and sampling facility served as reactor. The plant was operated in the downflow mode (i.e. with the flow direction from the top downward) with liquid circulation. The temperature was measured at the beginning (inlet) and at the end (outlet) of the catalyst bed by means of a thermocouple (cf. table below).

In the hydrogenation, a 40% strength by weight solution of distilled low-oligomer bisphenol A bisglycidyl ether (2,2-di[p-glycidoxyphenyl]propane, Epilox A 17-01 from Leuna-Harze, batch: 08/04, EEW=172 g/eq.) in stabilizer-free THF which comprised 4.5% by weight of water was used. The hydrogenation was carried out at a space velocity over the catalyst of 0.12 kg_{starting material}/L_{catalyst}•h, a temperature of about 43-45° C., a hydrogen pressure of 250 bar, a hydrogen feed rate of 25 standard l/h and a circulation of 3.1 kg/h over a period of 161 hours. A sample taken after 161 hours of operation was freed of solvent at 110° C. under reduced pressure (10 mbar) on a rotary evaporator and analyzed.

The conversion was 90% (H-NMR), and the epoxy equivalent weight was 209 g/equivalent, corresponding to a selectivity of 85%. The ruthenium content of the reactor output which had been freed of the solvent was 0.1 ppm.

Determination of Volatile Compounds (Extract from DIN 16945 4.8)

About 5 g of hydrogenated bisphenol A bisglycidyl ether (mass weighed in: ml) are weighed into a sheet metal lid having a flat bottom (75±5 mm diameter, with a rim height of about 12 mm) to a precision of 1 mg and, unless specified otherwise, stored at 140±2° C. for 3 hours in an oven. After cooling to room temperature, the material is weighed (final mass: fm).

The mass loss (=proportion of volatile compounds) in % is calculated as follows:

$$Mass loss = \frac{( m i - f m )}{m i} \cdot 100$$

The conversion and the degree of hydrogenation were determined by means of ¹H-NMR:
Amount of sample: 20-40 mg, solvent: CDCl₃, 700 μliter using TMS (tetramethylsilane) as reference signal, sample tube: 5 mm diameter, 400 or 500 MHz, 20° C.; decrease in the signals of the aromatic protons vs. increase in the signals of the aliphatic protons). The conversion reported in the examples is based on the hydrogenation of the aromatic groups.

The determination of the decrease of the epoxide groups was carried out by comparison of the epoxy equivalent weight (EEW) before and after hydrogenation, in each case determined in accordance with the standard ASTM-D-1652-88.

The determination of ruthenium in the output which had been freed of THF and water was carried out by means of mass spectrometry with inductively coupled plasma (ICP-MS, see below).

Oligomer Content:

According to the invention, it has also been recognized that the oligomer content of the feed has an influence on the operating life of the catalyst: when a distilled feed (“low-oligomer” feed) is used, a slower catalyst deactivation than in the case of a standard commercial product (“oligomer-rich” feed) is observed. The oligomer content can be determined, for example, by GPC measurement (gel permeation chromatography):

| | “Monomer” | “Oligomers” | |
|---|---|---|---|
| Product | 180-<380 g/mol | 380-<520 g/mol | 520-1500 g/mol |
| Standard | 89.98% by area | 2.05% by area | 7.97% by area |
| product | | | |
| Distilled | 98.80% by area | 0.93% by area | 0.27% by area |
| product | | | |

Molar mass of 2,2-di[p-glycidoxyphenyl]propane: 340 g/mol

Description of the GPC Measurement Conditions

Stationary phase: 5 styrene-divinylbenzene gel columns “PSS SDV linear M” (each 300×8 mm) from PSS GmbH (Temperature: 35° C.).

Mobile phase: THF (flow: 1.2 ml/min.).
Calibration: MW 500-10 000 000 g/mol using PS calibration kit from Polymer Laboratories. In the oligomer range: ethylbenzene/1,3-diphenylbutane/1,3,5-triphenylhexane/1,3,5,7-tetraphenyloctane/1,3,5,7,9-pentaphenyldecane.
Evaluation limit: 180 g/mol.
Detection: RI (index of refraction) Waters 410, UV (at 254 nm) Spectra Series UV 100.

The molar masses reported are, owing to different hydrodynamic volumes of the individual polymer types in solution, relative values based on polystyrene as calibration substance and are thus not absolute values.

The oligomer content in % by area determined by GPC measurement can be converted into % by weight by means of an internal or external standard.

GPC analysis of an aromatic bisglycidyl ether of the formula II (R═CH₃) used in the hydrogenation process of the invention showed, for example, apart from the monomer, the following content of corresponding oligomeric bisglycidyl ethers:

Molar masses in the range 180-<380 g/mol: >98.5% by area,
in the range 380-<520 g/mol: <1.3% by area,
in the range 520-<860 g/mol: <0.80% by area and
in the range 860-1500 g/mol: <0.15% by area.

Description of the Method of Determining the Evaporation Residue

About 0.5 g of each sample was weighed into a weighing bottle. The weighing bottles were subsequently placed at room temperature in a plate-heated vacuum drying oven and the drying oven was evacuated. At a pressure of 3 mbar, the temperature was increased to 200° C. and the sample was dried for 2 hours. The temperature was increased to 300° C. for a further 2 hours, and the samples were subsequently cooled to room temperature in a desiccator and weighed.

The residue (oligomer content) determined by this method on standard product (ARALDIT GY 240 BD from Vantico) was 6.1% by weight.

The residue (oligomer content) determined by this method on distilled standard product was 0% by weight. (Distillation conditions: 1 mbar, bath temperature 260° C., and temperature at which the distillate went over at the top 229° C.).

Determination of the cis/cis-cis/trans-trans/trans Isomer Ratios

A hydrogenated bisphenol A bisglycidyl ether (R═CH₃) product mixture was analyzed by means of gas chromatography (GC and GC-MS). 3 signals were identified as hydrogenated bisphenol A bisglycidyl ether.

The hydrogenation of the bisphenol A unit of the bisglycidyl ether can result in a plurality of isomers. Depending on the arrangement of the substituents on the cyclohexane rings, cis/cis, trans/trans or cis/trans isomerism can occur.
To identify the three isomers, the products of the peaks in question were collected preparatively by means of a column arrangement. Each fraction was subsequently characterized by NMR spectroscopy (¹H, ¹³C, TOCSY, HSQC).

For the preparative GC, a GC system having a column arrangement was used. In this system, the sample was subjected to preliminary separation on a Sil-5 capillary (I=15 m, ID=0.53 mm, df=3 μm). The signals were cut onto a 2nd GC column with the aid of a DEANS connection. This column served to check the quality of the preparative fraction. Each peak was subsequently collected by means of a fraction collector. 28 injections of an about 10% strength by weight solution of the sample was prepared, which corresponds to about 10 μg of each component.

The isolated components were then characterized by NMR spectroscopy.

For the determination of the isomer ratios of a hydrogenated bisphenol F bisglycidyl ether (R═H), an analogous method is used.

Determination of Ruthenium in the Ring-Hydrogenated Bisglycidyl Ether of the Formula I

The sample was diluted by a factor of 100 with a suitable organic solvent (e.g. NMP). The ruthenium content of this solution was determined by mass spectrometry with inductively coupled plasma (ICP-MS).

Instrument: ICP-MS spectrometer, e.g. Agilent 7500s

Measurement conditions:

| | |
|---|---|
| Calibration: | External calibration in organic matrix |
| Atomizer: | Meinhardt |
| Mass: | Ru102 |

The calibration curve was selected so that the necessary specification value could be determined reliably in the diluted measurement solution.

Determination of Chloride and Organically Bound Chlorine

The determination of chloride was carried out by ion chromatography.

Sample preparation:
About 1 g of the sample was dissolved in toluene and extracted with 10 ml of high-purity water. The aqueous phase was analyzed by means of ion chromatography.
Measurement conditions:

| | |
|---|---|
| Ion chromatography system: | Metrohm |
| Precolumn: | DIONEX AG 12 |
| Separation column: | DIONEX AS 12 |
| Eluent: | (2.7 mmol of Na₂CO₃ + 0.28 |
| | mmol of NaHCO₃)/liter of water |
| Flow: | 1 ml/min. |
| Detection: | conductivity after chemical |
| | suppression |
| Suppressor: | Metrohm module 753 |
| | 50 mmol of H₂SO₄; high-purity |
| | water (flow about 0.4 ml/min.) |
| Calibration: | 0.01 mg/l to 0.1 mg/l |

Coulometric determination of organically bound chlorine (total chlorine), corresponding to DIN 51408, Part 2, “Bestimmung des Chlorgehalts”

The sample was burned in an oxygen atmosphere at a temperature of about 1020° C. As a result, the chlorine bound in the sample is converted into hydrogen chloride. The nitrous gases, sulfur oxides and water formed in the combustion are removed and the combustion gas which has been purified in this way is introduced into the coulometer cell. Here, the chloride formed is determined coulometrically according to

Cl⁻+Ag⁺→AgCl .

Sample weight range: 1 to 50 mg
Determination limit: about 1 mg/kg (substance-dependent)

### Instrument: Euroglas (LHG), “ECS-1200”

Literature: F. Ehrenberger, “Quantitative organische Elementaranalyse”, ISBN 3-527-28056-1.

1. A heterogeneous ruthenium catalyst comprising amorphous silicon dioxide as a support material, wherein the ruthenium catalyst is produced by a process comprising single or multiple impregnation of the silicon dioxide support material with a solution of a ruthenium salt, drying and reduction, wherein the silicon dioxide support material has a BET surface area of from 250 to 400 m²/g as determined according to DIN 66131, a pore volume of from 0.7 to 1.1 ml/g as determined according to DIN 66134, and a pore diameter of from 6 to 12 nm as determined according to DIN 66134.

2. The ruthenium catalyst according to claim 1, wherein the silicon dioxide support material has a BET surface area of from 290 to 370 m²/g.

3. The ruthenium catalyst according to claim 1, wherein the silicon dioxide support material has a pore volume of from 0.75 to 1.0 ml/g.

4. The ruthenium catalyst according to claim 1, wherein the silicon dioxide support material has a pore diameter of from 8 to 10 nm.

5. The ruthenium catalyst according to claim 1, wherein the catalyst comprises from 0.5 to 4% by weight of ruthenium, based on the weight of the silicon dioxide support material.

6. (canceled)

7. The ruthenium catalyst according to claim 1, wherein the single or multiple impregnation of the silicon dioxide support material is carried out in the presence of an aqueous solution of ruthenium(III) acetate.

8. The ruthenium catalyst according to claim 1, wherein the silicon dioxide support material is in the form of spherical shaped bodies for producing the catalyst.

9. The ruthenium catalyst according to claim 8, wherein the spherical shaped bodies have a diameter of from 3 to 5 mm.

10. The ruthenium catalyst according to claim 8, wherein the spherical shaped bodies have a lateral compressive strength of >60 N.

11. The ruthenium catalyst according to claim 1, further comprising less than 0.05% by weight of halide as measured by ion chromatography, based on the total weight of the ruthenium catalyst.

12. The ruthenium catalyst according to claim 1, wherein the ruthenium is concentrated as a shell at the catalyst surface.

13. The ruthenium catalyst according to claim 12, wherein the ruthenium in the shell is partly or wholly crystalline.

14. The ruthenium catalyst according to claim 1, wherein the ruthenium is present in finely dispersed form.

15. The ruthenium catalyst according to claim 14, wherein ruthenium dispersity is from 30 to 60% as measured by CO sorption in accordance with DIN 66136-3.

16. The ruthenium catalyst according to claim 1, wherein the total concentration of Al(III) and Fe(II and/or III) in the silicon dioxide support material is less than 300 ppm by weight.

17. A process for hydrogenating a carbocyclic aromatic group to form the corresponding carbocyclic aliphatic group in the presence of a heterogeneous ruthenium catalyst according to claim 1.

18. The process according to claim 17, for hydrogenating a benzene ring to form the corresponding carbocyclic 6-membered ring.

19. The process according to claim 17, for preparing a bisglycidyl ether of the formula I 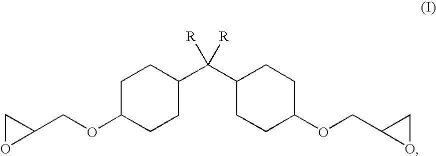
where R is CH₃ or H, by ring hydrogenation of the corresponding aromatic bisglycidyl ether of the formula II

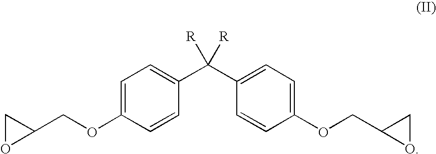

20. The process according to claim 19, wherein the aromatic bisglycidyl ether of the formula II has a content of corresponding oligomeric bisglycidyl ethers of less than 10% by weight.

21. (canceled)

22. The process according to claim 20, wherein the oligomeric bisglycidyl ethers have a molecular weight of from 568 to 1338 g/mol when R═H and have a molecular weight of from 624 to 1478 g/mol when R═CH₃.

23. The process according to claim 17, wherein the hydrogenation is carried out at a temperature of from 30 to 200° C.

24. The process according to claim 17, wherein the hydrogenation is carried out at an absolute hydrogen pressure of from 10 to 325 bar.

25. The process according to claim 17, wherein the hydrogenation is carried out over a fixed bed of catalyst.

26. The process according to claim 17, wherein the hydrogenation is carried out in a liquid comprising the catalyst in the form of a suspension.

27. The process according to claim 19, wherein the aromatic bisglycidyl ether of the formula II exists within a solution further comprising an organic solvent, which is inert toward the hydrogenation, and from 0.1 to 10% by weight water, based on the organic solvent.
